(19) United States
(12) Patent Application Publication
(10) Pub. No.: US 2008/0125340 A1
(43) Pub. Date: May 29, 2008

(54) Personal cleansing composition with enhanced skin feel characteristics

(75) Inventor: Robert Dail, Phoenix, AZ (US)

Correspondence Address: THE DIAL CORPORATION, 15501 N. DIAL BOULEVARD, SCOTTSDALE, AZ, 85260, US

(73) Assignee: The Dial Corporation

(21) Appl. No.: 12/011,292

(22) Filed: Jan. 25, 2008

Related U.S. Application Data
(63) Continuation-in-part of application No. 11/465,581, filed on Aug. 18, 2006.
(60) Provisional application No. 60/709,718, filed on Aug. 19, 2005.

(51) Int. Cl. A61K 8/25; A61Q 19/10

(52) U.S. Cl. 510/130

(57) ABSTRACT

Personal cleansing compositions with enhanced skin feel characteristics are provided. In accordance with an exemplary embodiment, a personal cleansing composition includes: (i) about 1% to about 6% by weight of the personal cleansing composition (WPCC) of talc, and (ii) about 3% to about 10% by WPCC of free fatty acid (FFA). The personal cleansing composition is substantially free of non-FFA additive skin refatting agents.

### CROSS-REFERENCES TO RELATED APPLICATIONS

The present application is a continuation-in-part of U.S. application Ser. No. 11/465,581, filed Aug. 18, 2006, which claims the benefit of U.S. Provisional Application No. 60/709,718, filed Aug. 19, 2005, which is herein incorporated in its entirety by reference.

### TECHNICAL FIELD

The present invention relates generally to personal care cleansing compositions and, more particularly, to personal care cleansing compositions including unique combinations of filler (e.g., talc) and free fatty acid that provide enhanced skin feel characteristics.

### BACKGROUND

Personal care compositions, such as toilet soap bars, generally contain one or more soaps (i.e., monovalent salts of monocarboxylic fatty acids), synthetic detergents, or a combination of synthetic detergents and soaps (as in the case of combars). If the personal care composition contains one or more soaps, the soaps may each include: (i) a long fatty acid chain having alkyl chain links ranging from about 8 carbons to about 18 carbons in length, and (ii) one or more types of counter-ions, such as sodium ions, potassium ions, ammonium, alkanol ammonium, and the like. The personal care composition is often further provided with a number of additives that impart desirable characteristics to the composition not inherent in the soap itself. Moisturizers, for example, may be added to treat skin dryness; humectants may be added to promote moisture retention; antibacterial agents may be added for disinfection purposes; mixtures of synthetic oils may be added for fragrance; dyes may be added for color; etc.

Of the many attributes that render a personal cleansing composition appealing to consumers, agreeable skin feel characteristics are undoubtedly amongst the most important. As appearing herein, the term “skin feel characteristics” is defined to include various attributes appreciable to a user during and after application of the personal care composition, typically in a wet state, to the skin. These attributes may include, but are not limited to, the degree to which the user's skin feels refreshed, softened, and/or moisturized during and after use of the personal care composition; the perceived mildness of the personal care composition; the perceived quality (e.g., creaminess) of the lather and foam; the lack or minimization of skin irritation; and the tactile quality of any residue remaining on the skin after rinsing and drying, both immediately after rinsing and drying and subsequent to a predetermined time period (e.g., 30 minutes).

Soap manufacturers have long recognized the importance of providing consumers with personal cleansing compositions having desirable skin feel characteristics. Accordingly, to enhance skin feel characteristics, personal cleansing compositions typically do not contain skin irritants, such as, for example, grease solvents. Furthermore, it has become common practice to add small amounts of free fatty acids (FFAs) to the personal cleansing composition to improve the quality of lather, to increase the production of foam, and to otherwise enhance the composition's overall skin feel characteristics. However, the addition of free fatty acids, alone, has not been found to provide an adequate improvement in skin feel characteristics. For this reason, many soap manufacturers further enhance the skin feel characteristics of their toilet soap bars through the utilization of various non-FFA additive skin refatting agents (e.g., emollients, humectants, occulsives, etc.). As appearing herein, the term “additive skin refatting agents” is defined to exclude refatting agents that are produced as a byproduct of the saponification process, such as glycerin. Examples of such additive skin refatting agents include water-insoluble silicones, petrolatum, high molecular weight polyethylene glycols, and vegetable triglycerides (e.g., cocoa butter, shea butter, etc.). Although effective at enhancing the skin feel characteristics of a personal cleansing composition, the addition of such non-FFA additive skin refatting agents are associated with a number of disadvantages, including high cost and lather suppression.

Accordingly, it is desirable to provide personal cleansing compositions having enhanced skin feel characteristics and that are substantially free of non-FFA additive skin refatting agents (e.g., emollients, humectants, occulsives, etc.). In addition, it is desirable to provide personal cleansing compositions that are relatively inexpensive to produce. Other desirable features and characteristics of the present invention will become apparent from the subsequent detailed description of the invention and the appended claims, taken in conjunction with this background of the invention.

### BRIEF SUMMARY

A personal cleansing composition with enhanced skin feel characteristics is provided in accordance with an exemplary embodiment of the present invention. The personal cleansing composition includes: (i) about 1% to about 6% by weight of the personal cleansing composition (WPCC) of talc, and (ii) about 3% to about 10% by WPCC of free fatty acid (FFA). The personal cleansing composition is substantially free of non-FFA additive skin refatting agents.

A personal cleansing composition with enhanced skin feel characteristics is provided in accordance with another exemplary embodiment of the present invention. The personal cleansing composition includes: (i) about 1% to about 6% by WPCC of total fillers, and (ii) about 3% to about 10% by WPCC of total additive skin refatting agents. At least 50% of the total fillers consists of talc.

A personal cleansing composition with enhanced skin feel characteristics is provided in accordance with a further exemplary embodiment of the present invention. The personal cleansing composition includes: (i) about 4% to about 5% by WPCC of talc, and (ii) about 5% to about 8% by WPCC of FFA.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present inventor has discovered that, when certain quantities of total fillers, particularly talc, are combined with certain quantities of free fatty acids (FFAs) within a personal care composition, an unexpected and dramatic improvement in the skin feel characteristics of the resulting personal care composition is achieved. As previously explained, the term “skin feel characteristics” is defined herein to include various attributes appreciable to a user during and after application of the personal care composition, typically in a wet state, to the skin. Examples of these attributes are set-forth above in the preceding section entitled “Background.”

The enhanced skin feel characteristics are exhibited over a quantitative range of fillers (e.g., talc) and FFAs. Testing data reveals that statistically-significant benefits in skin feel characteristics first appear at quantities of about 1% by weight of the personal cleansing composition (WPCC) of filler and significantly diminish, or entirely disappear, at quantities exceeding about 6% by WPCC of filler. Similarly, statistically-significant benefits in skin feel characteristics first appear at quantities of about 1% by WPCC of FFA. Furthermore, if the personal care composition assumes the form of soap bar, the soap bar may become undesirably malleable at quantities exceeding about 10% by WPCC of FFA. Still more surprisingly, skin feel characteristics were found to be especially pronounced in a preferred embodiment of the composition containing about 3% to about 5% by WPCC of filler and about 3% to about 8% by WPCC of FFA. In a preferred embodiment, the personal care composition contains about 4% to about 5% by WPCC of filler combined with about 5% to about 8% by WPCC of FFA. In a still more preferred embodiment wherein the most pronounced improvements in skin feel characteristics were observed, the personal care composition contains about 5% by WPCC of talc and about 5% by WPCC of FFA. The foregoing quantities are based on substantially 100% active components.

The perceived benefits in skin feel characteristics do not share a linear relationship with increased quantities of fillers (e.g., talc) and FFAs. That is, skin feel characteristics do not ostensibly improve by simply increasing the quantities of fillers contained within the personal care composition over a large range; little to no improvement in skin feel characteristics are observed when about 1% to about 10% by WPCC of FFA is combined with more than about 6% by WPCC of filler, and a negative impact in skin feel characteristics is seen when about 1% to about 10% by WPCC of FFA is combined with more than about 10% by WPCC of filler. This non-linearity between the quantity of FFA, the quantity of total filler, and the perceived benefits in skin feel characteristics is indicative of the surprising nature of the present inventor's discovery, namely, that drastic improvements in skin feel characteristics may be achieved over a specific quantitative range of FFAs when combined with a specific quantitative range of total fillers, particularly talc.

Free Fatty Acid Components

As used herein, the term “fatty acid” is defined to include a molecule comprising an aliphatic chain having a carboxylic acid group bonded to an end thereof. As is well-known, a fatty acid may generally be described by the formula CH₃(CH₂)ₙCOOH, wherein n is typically an even number. The term “free fatty acids” is, in turn, defined in a broad, but traditional sense to describe fatty acids that are, in large part, not bound to other molecules. In a preferred embodiment, all or any of the free fatty acids are derived from tallow, coconut oil, palm kernel oil, palm oil (including palm stearin oil), babassu oil, and/or hydrogenates of the foregoing compositions. In still further embodiments, the FFA may comprise any of the following: arachidic acid, arachidonic acid, beeswax acid, behenic acid, C10-40 hydroxyalkyl acid, C10-40 isoalkyl acid, C32-36 isoalkyl acid, corn acid, cottonseed acid, erucic acid, hydrogenated menhaden acid, hydroxystearic acid, isomerized linoleic acid, isomerized safflower acid, isostearic acid, linseed acid, olive acid, peanut acid, pelargonic acid, rapeseed acid, rice bran acid, ricinoleic acid, safflower acid, soy acid, sunflower seed acid, tall oil acid, undecanoic acid, undecylenic acid, and wheat germ acid.

The above-described improvement in skin feel characteristics is significant enough to drastically reduce or entirely eliminate the need for large quantities of expensive non-FFA additive skin refatting agents (e.g., emollients, humectants, occulsives, etc.), which traditionally have been thought necessary to yield adequately improved skin feel characteristics. Accordingly, in an exemplary embodiment of the present invention, the personal cleansing composition is substantially free of non-FFA additive skin refatting agents. As appearing herein, the term “substantially free of non-FFA additive skin refatting agents” means that, if present, such non-FFA additive skin refatting agents exist in an amount below that which would have a significant perceivable effect on the skin feel characteristics of the personal cleansing composition as shown by, for example, statistical testing. In a preferred embodiment, the personal cleansing composition contains less than about 0.5% by WPCC of non-FFA additive skin refatting agents and, more preferably, about 0% by WPCC of non-FFA additive skin refatting agents. Again, additive skin refatting agents are defined to exclude substances produced as a byproduct of the saponification process, including, for example, glycerin. The elimination or reduction of non-FFA additive skin refatting agents may also be described in terms of the total skin refatting agents contained within the personal cleansing composition. For example, an embodiment of the inventive personal cleansing composition can be described as containing about 1% to about 10%, and preferably about 5% to about 8%, by WPCC of total additive skin refatting agents, wherein the total additive skin refatting agents consist substantially entirely of free fatty acid.

Filler Components

A wide variety of inert materials, both organic and inorganic, are suitable for use as filler in the above-described compositions. Such materials include, but are not limited to, talc, fumed silica, starch, and various combinations of the foregoing materials. In one embodiment, the filler comprises one or more minerals, such as clay or talc. In a still more preferred embodiment, the filler consists of more than 50% by WPCC of talc and, more preferably, consists substantially entirely of talc. As appearing herein, the term “talc” denotes a composition consisting entirely or almost entirely of hydrated magnesium silicate. Talc may generally be described by either of the following formulas: H₂Mg₃(SiO₃)₄ or Mg₃Si₄O₁₀(OH)₂. Preferably, talc in the form of talcum powder is utilized. Talc is the preferred filler due, at least in part, to its lubricating effects and to its ability to increase the overall moisture content of the personal care composition. The addition of talc results in enhancement of a number of skin feel characteristics (e.g., noticeably creamier lather). Without intending to be bound by theory, this is believed to be the result of an increase in the surface area of the personal care composition, in bar form, when granules of talc are separated from the bar thus producing tiny cavities in the bar's outer surface into which water may flow. In an exemplary embodiment, the personal cleansing composition contains less than about 0.5% by WPCC of non-talc fillers and, preferably, about 0% by WPCC of non-talc fillers.

Additional Components

In accordance with exemplary embodiments of the present invention, the above-described personal cleansing composition should contain one or more soaps as traditionally defined (i.e., monovalent salts of monocarboxylic fatty acids), synthetic detergents, or a combination of soaps and synthetic detergents. In addition, the personal cleansing composition may also include various additives well-known in the industry. A non-exhaustive list of such additives includes moisturizers, humectants, antibacterial agents, water, polymers, dyes, fragrances, preservatives, pH adjusters, and the like. The addition of quaternary ammonium compounds (i.e., long chain alkylamidopropyl ethyldimonium ethosulfates having alkyl groups derived from various sources) may also enhance the skin feel characteristics of the resulting personal cleansing composition. Personal care compositions including such quaternary ammonium compounds are described in detail in U.S. application Ser. No. 11/465,581, filed Aug. 18, 2006.

A Non-Limiting Example of a Personal Cleansing Composition Formula

By way of illustration and not of limitation, the formulation of one exemplary personal cleansing composition is set-forth in the following table.

| Component | Weight % |
|---|---|
| Sodium Soap | 71.5 |
| Water | 12.1 |
| Free Fatty Acid | 5.0 |
| Talc | 5.0 |
| Perfume | 1.4 |
| Glycerin | 0.9 |
| Dye(s) | 0.016 |
| Other (e.g., salt, triclocarbon, etc.) | 4.084 |

Accordingly, various exemplary embodiments of personal cleansing compositions having enhanced skin feel characteristics have been provided. In one exemplary embodiment, the personal cleansing compositions comprise preferably about 1% to about 6%, more preferably about 3% to about 5%, and still more preferably about 4% to about 5% by WPCC of talc. In a further exemplary embodiment, the personal cleansing compositions comprise about 1% to about 10%, and preferably about 5% to about 8%, by WPCC of free fatty acid. In a still further exemplary embodiment, the personal care cleansing compositions are substantially free of non-FFA additive skin refatting agents (e.g., emollients, humectants, occulsives, etc.). Due to the reduction or elimination of non-FFA additive skin refatting agents, and due to the addition of filler, embodiments of the above-described cleansing compositions are relatively inexpensive to produce.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A personal cleansing composition with enhanced skin feel characteristics, the personal cleansing composition comprising:
about 1% to about 6% by weight of the personal cleansing composition (WPCC) of talc; and
about 1% to about 10% by WPCC of free fatty acid (FFA);
wherein the personal cleansing composition is substantially free of non-FFA additive skin refatting agents.

2. The personal cleansing composition according to claim 1, comprising less than about 0.5% by WPCC of non-FFA additive skin refatting agents.

3. The personal cleansing composition according to claim 2, comprising about 0% by WPCC of non-FFA additive skin refatting agents.

4. The personal cleansing composition according to claim 1, comprising about 3% to about 10% by WPCC of FFA.

5. The personal cleansing composition according to claim 4, comprising about 5% to about 8% by WPCC of FFA.

6. The personal cleansing composition according to claim 1, comprising about 3% to about 5% by WPCC of talc.

7. The personal cleansing composition according to claim 6, comprising about 4% to about 5% by WPCC of talc.

8. The personal cleansing composition according to claim 1, comprising:
about 5% by WPCC of talc; and
about 5% by WPCC of FFA.

9. The personal cleansing composition according to claim 1, comprising less than about 0.5% by WPCC of non-talc fillers.

10. The personal cleansing composition according to claim 9, comprising about 0% by WPCC of non-talc fillers.

11. A personal cleansing composition with enhanced skin feel characteristics, the personal cleansing composition comprising:
about 1% to about 6% by weight of the personal cleansing composition (WPCC) of total fillers; and
about 1% to about 10% by WPCC of total additive skin refatting agents;
wherein at least 50% of the total fillers consists of talc.

12. The personal cleansing composition according to claim 11, wherein the total fillers consist substantially entirely of talc.

13. The personal cleansing composition according to claim 12, wherein the total additive skin refatting agents comprise less than about 0.5% by WPCC of non-FFA additive skin refatting agents.

14. The personal cleansing composition according to claim 12, wherein the total additive skin refatting agents are substantially free of non-FFA additive skin refatting agents.

15. The personal cleansing composition according to claim 12, comprising:
about 3% to about 5% by WPCC of talc; and
about 3% to about 10% by WPCC of free fatty acid.

16. A personal cleansing composition with enhanced skin feel characteristics, the personal cleansing composition comprising:
about 4% to about 5% by weight of the personal cleansing composition (WPCC) of talc; and
about 5% to about 8% by WPCC of free fatty acid.

17. The personal cleansing composition according to claim 16, comprising:
less than about 0.5% by WPCC of non-FFA additive skin refatting agents; and
less than about 0.5% by WPCC of fillers other than talc.

18. The personal cleansing composition according to claim 17, comprising:
about 0% by WPCC of non-FFA additive skin refatting agents; and
about 0% by WPCC of fillers other than talc.
